(19) United States
(12) Patent Application Publication
(10) Pub. No.: US 2022/0207128 A1
(43) Pub. Date: Jun. 30, 2022

(54) DATA SHARING

(71) Applicant: NumberEight Technologies Ltd, London (GB)

(72) Inventors: Abhishek Sen, London (GB); Christopher John Watts, London (GB)

(21) Appl. No.: 17/601,696

(22) PCT Filed: Apr. 6, 2020
(86) PCT No.: PCT/GB2020/050902

§ 371 Date: Oct. 5, 2021

(30) Foreign Application Priority Data
Apr. 5, 2019 (GB) 1904894.1

(51) Int. Cl. G06F 21/44; G06F 21/60; G06F 21/62; G06F 21/64; G06F 11/34

(52) U.S. Cl. CPC G06F 21/44; G06F 21/606; G06F 11/3438; G06F 21/64; G06F 21/6218

(57) ABSTRACT

The present disclosure relates to an apparatus for sharing data, the apparatus comprising: means for receiving data signals; wherein the data signals include a plurality of types of contextual information relating to a user, wherein each type of contextual information comprises information inferred from sensor data; and means for performing an operation in dependence on the plurality of types of contextual information.

### FIELD OF THE DISCLOSURE

The present disclosure relates to data sharing. The disclosure provides a method of sharing data signals containing contextual information, in particular it provides a method of sharing data signals containing contextual information relating to a user, the contextual information having been inferred.

### BACKGROUND TO THE DISCLOSURE

Devices, such as phones, watches, and computers, often contain a data processing capability that is adapted to store or analyse information relating to the user of the device. As an example, devices might monitor and store the heart rate of the user. These devices normally work in isolation, where the interactions of the user with each device are separate and disconnected from one another.

### SUMMARY OF THE DISCLOSURE

As used herein, ‘event’ preferably connotes an identifiable occurrence. Typically, an event refers to a noticeable change in a property, such as: a user moving between locations; a threshold value, e.g. a threshold stress level, being exceeded; a time condition being met (e.g. it being dawn); and the receipt or transmission of data. An event may occur without input from a user, such as a change in temperature, or an event may involve an action taken by a user, such as pressing a button or saying a word.

As used herein, ‘contextual information’ preferably connotes information that relates to the context of a user and/or information that is inferred. Typically, the information is inferred from sensor data and/or from information received from other devices. Examples of inferring information include: inferring a stress level from heart rate data; inferring a location from a time and a user's historic behaviour; and inferring a time of day (e.g. dawn/dusk) from a time and an ambient light level.

As used herein, ‘context’ preferably connotes information that refers to the activity, actions, and interactions of a user, for example whether a user is undertaking a specific action, whether a user has indicated an intent to undertake a specific action, and/or whether a user is, or will be, in certain surroundings.

As used herein, ‘action’ preferably connotes a physical action taken by a user; optionally, a physical action may be considered distinct from an interaction with a device.

As used herein, ‘inferring’ preferably connotes determining information from at least one datum—where the determined information is different from the information contained in the datum. Preferably, but not exclusively, inferring relates to the determination of a different type of information to that contained in the datum. Inferring may include the use of formulae, databases, reference lists, neural networks, and machine learning techniques and methods.

Examples of inference include determining a weather from a temperature; determining a mood from a heart rate; and determining an activity from a velocity. Typically, inferring involves consideration of data (as opposed to a single datum).

According to at least one aspect of the disclosure herein, there is provided an apparatus for sharing data, the apparatus comprising: means for receiving data signals; where the data signals include contextual information relating to a user; and means for performing an operation in dependence on the contextual information. The sharing of contextual information enables the performance of operations in dependence on the context of the user, which would not otherwise be possible. In particular, the sharing of contextual information is useable to share information relating to a current status of the user. This current status may then be used to determine a previous time where the user had a similar status and operations may be performed in dependence on operations selected by the user at that previous time.

The means for receiving data signals is typically a communications interface, such as a Bluetooth® receiver, an Ethernet interface, or a 3G, 4G, or 5G interface. The means for receiving data signals may comprise a receiving module and/or a processor that is arranged to receive sensor data and to infer contextual information from that sensor data before the data signals containing the contextual information are provided to the means for performing an operation. The means for performing an operation is typically a processor or a circuit—this means may be implemented in hardware or software or a combination of hardware and software.

According to at least one aspect of the disclosure herein, there is provided an apparatus for sharing data, the apparatus comprising: means for receiving data signals; where the data signals include a plurality of different types of contextual information relating to a user, wherein each type of contextual information comprises information inferred from sensor data; and means for performing an operation in dependence on the plurality of different types contextual information.

The different types of contextual information typically comprise different contextual information referring to different aspects of a user's context, for example the types of contextual information may include a mood, a weather (e.g. warm and humid), or an activity, where each of these types of contextual information is inferred from sensor data.

Sensor data may comprise any data that is initially recorded by a sensor, for example a temperature recorded by a temperature sensor or a heart rate recorded by a heart rate sensor. Sensor data may be processed before the inference, where this inference is therefore still based on sensor data (albeit processed sensor data). Inferences may also be based on already inferred information, where this already inferred information is based on sensor data (e.g. a mood may be inferred from an activity, which may be inferred from a heart rate—where the heart rate is obtained using sensor data).

The second inference is therefore based on the initial sensor data. Examples of sensor data, and sensors, include GPS being used to measure locations, accelerometers being used to measure the orientation and movement of a device, and communications sensors being used to identify communications networks.

The apparatus may comprise an application on a device so that receiving data signals containing the contextual information may comprise receiving data signals from another application on the same device. Therefore, the sensor information may be received by a first application, the contextual information may be inferred by a second application, and the operation may be performed by a third application, where each of the first, second, and third application are on the same device.

### Contextual Information

Preferably, the contextual information comprises information relating to an attribute of the user. The attribute of the user may be inferred from information relating to: the user's environment; and the user's interactions with devices.

Preferably, the contextual information comprises information relating to a physical attribute of the user, such as: the activity of the user; the location of the user; and the engagement with devices of the user; and properties of persons in proximity to the user; a psychological attribute of the user, such as the intentions of the user; the mindset of the user; and the mood and/or stress level of the user; a current attribute of the user; and/or a historic attribute of the user, such as the previous activity of the user; and/or the previous behaviour of the user.

Preferably, the contextual information comprises information relating to historic activities, such as previous operations that have been performed.

Optionally, the contextual information comprises information relating to an attribute of the environment and/or the apparatus.

Preferably, the contextual information comprises information inferred from data, preferably wherein information is inferred indirectly and/or wherein the data is raw data. For example, contextual information may be inferred from sensor data determined using a sensor of the apparatus.

Optionally, the data includes at least one of: a temperature; a heart rate; an orientation; a speed; an acceleration; and a location.

Preferably, the contextual information comprises information inferred from a plurality of data, preferably wherein information is inferred from a plurality of types of data and/or a plurality of data from different sources. The determination of contextual information from a plurality of sources enables a more varied range of contextual information to be determined than would be possible using a single source of data.

### Permissions

Preferably, the apparatus further comprises means for determining a permission relating to the apparatus wherein the permission indicates whether the apparatus is permitted to receive and/or access a type of contextual information.

Preferably, the means for determining a permission is arranged to determine a plurality of permissions, wherein each permission relates to a different type of contextual information.

Preferably, the apparatus further comprises means for determining a permission relating to the apparatus wherein the permission indicates whether the apparatus is permitted to infer a type of contextual information.

Preferably, the apparatus is arranged to use sensor data to infer contextual information, wherein the types of contextual information that the apparatus is arranged to infer depending on the permission.

Preferably, the apparatus further comprises means for determining a permission relating to the apparatus wherein the permission indicates whether the apparatus is permitted to receive and/or access a type of sensor data.

Preferably, the means for determining a permission is arranged to determine a plurality of permissions, wherein each permission relates to a different type of sensor data.

Optionally, the means for determining a permission comprises a processor.

The use of permissions enables contextual information to be inferred and communicated based on a user preference, where the user may wish to treat different types of contextual information differently (e.g. some types of contextual information may be considered more sensitive than other types).

### Contextual Information—Location of Transmission

Optionally, the plurality of data is obtained solely by the apparatus, preferably by a number of sensors of the apparatus. Obtaining data using solely the apparatus avoids sharing potentially sensitive information with other apparatuses.

Preferably, the apparatus comprises at least one sensor, wherein the sensor is arranged to obtain the sensor data.

Preferably, the apparatus comprises a plurality of sensors, wherein the sensors are arranged to obtain a plurality of different types of sensor data.

Preferably, the apparatus is arranged to receive sensor data from one or more further apparatuses.

Preferably, the plurality of data is obtained by a plurality of applications, preferably wherein at least one of the plurality of applications is separate from and/or external to the apparatus. By obtaining data from a plurality of applications, in particular applications external to the apparatus, data can be obtained that would not otherwise be available to the apparatus—for example data can be obtained from further apparatuses in use at times when the apparatus was not in use.

Preferably, the plurality of applications are on a plurality of apparatuses.

Preferably, the means for receiving data signals is adapted to receive data from a database comprising contextual information. Optionally, the apparatus further comprises means for storing a database comprising contextual information. The use of a database containing contextual information enables information to be obtained at any time, for example information can be obtained from apparatuses that are not able to transmit data when the data is desired. The use of a database also allows quick access to data and avoids the need to download data at the time of querying data.

Preferably, the means for receiving data signals is adapted to receive data signals from a transmitting application, preferably wherein the transmitting application comprises a sensor not included in the apparatus.

Preferably, the transmitting application is located on a separate apparatus.

Preferably, the apparatus comprises an ambient device and the separate apparatus comprises a personal device and/or the apparatus comprises a personal device and the separate apparatus comprises an ambient device. An ambient device is typically a device that is not regularly moved, whereas a personal device is typically a device that accompanies a user as the user moves. Personal devices typically gather information relating to the user which is not available to ambient devices. Conversely, ambient devices may have more sophisticated sensors than ambient devices. The sharing of information between these types of devices ensures that each device has access to a large range of information.

Optionally, the means for receiving data signals is adapted to receive data signals from the transmitting application via an intermediary application. As is the case when using a database, this enables the receipt of data regardless of the status of the transmitting application. This also enables the use of an intermediary application that collates data and is thus able to provide a greater variety of contextual information.

Preferably, the apparatus further comprises means for updating a database comprising contextual information. The means for updating may comprises means for transmitting data signals. By updating a database, the apparatus is adapted to provide contextual information that may thereafter be received (and used) by other apparatuses.

The means for updating a database typically comprises a processor and/or a circuit. It may also comprise a transmitting device, such as an internet interface, a Bluetooth® interface or a 3G, 4G, or 5G interface.

### Context History

Preferably, the apparatus further comprises means for forming a context history, preferably based on the received contextual information in the received data signals. The formation of a context history enables the apparatus to compare contextual information relating to the present status of the user to previous contextual information in order to suggest appropriate operations based on historic data, such as the historic behaviour of the user and historic operations performed when the user had a similar status.

Preferably, the means for forming a context history operation comprises a neural network. The forming of a contextual history may also comprise boosted decision trees, Markov methods, and other machine learning techniques.

Preferably, the apparatus further comprises means for receiving further data signals, the further data signals containing historic contextual information; and means for forming a context history based on the historic contextual information.

The means for receiving further data signals may be similar to the means for receiving data signals, e.g. it may be a communications interface. The means for forming a context history is typically similar or the same as the means for performing an operation, e.g. a processor or a circuit.

Preferably, the context history comprises at least one of: previous contextual information; information relating to the previous actions of the user during a certain time period; information relating to the previous actions of the user relating to a context; information relating to the previous actions of the user relating to a mood. The forming of the context history may also comprise consideration of any or all of this information.

Preferably, the apparatus comprises means for determining the context history based on a plurality of data signals received at different times, preferably wherein the data signals are received at least one of: at least ten seconds apart; at least a minute apart; at least thirty minutes apart; at least an hour apart; at least a day apart; at least a week apart; at least a month apart; at least a year apart; and at least five years apart. The context history may be formed using data received over a period of history and/or may be periodically or continuously updated. This enables a present piece of contextual information to be compared to the context history to determine a similarity and/or a difference between the present contextual information and the historic contextual information.

Determination of the context history may also be based on contextual information received from different applications, devices, and/or apparatuses.

Preferably, the apparatus further comprises means for comparing the received contextual information in the received data signals to historic contextual information in the context history. The means for forming comparing is typically similar or the same as the means for performing an operation, e.g. a processor or a circuit.

Preferably, the apparatus further comprising means for determining a baseline value for a type of contextual information relating to the user; and determining a difference between a recent value for the type of contextual information and the baseline value; where performing an operation in dependence on the contextual information comprises performing an operation in dependence on the determined difference. This operation enables variation from a baseline value to be determined and actions to be determined based upon this variation. As an example a variation from a relaxed state may be used to determine that the user is stressed. The means for forming determining a baseline is typically similar or the same as the means for performing an operation, e.g. a processor or a circuit.

According to another aspect of the disclosure herein, there is provided an apparatus for sharing data, the apparatus comprising: means for receiving data signals; wherein the data signals include contextual information relating to a user; means for comparing the received contextual information to historic contextual information; and means for performing an operation in dependence on the comparison.

Each means may be similar to the means described with relation to any other apparatus described herein, e.g. the means for comparing may be a processor or a circuit.

Sharing Criteria (e.g. Sharing Based on an Event)

Preferably, the means for receiving data signals is adapted to receive data signals at a predetermined time. This may comprise the means for receiving being adapted to receive data signals at a certain time each day, week, or month, or being adapted to receive data signals after a set time period has elapsed following a prior receipt.

Optionally, the means for receiving data signals is adapted to receive data signals at least one of: at least once every month; at least once every week; at least once every day, at least once every hour; at least once every minute; at least once every second; and substantially continuously.

Preferably, the apparatus is arranged to receive data signals from and/or send data signals to a further, preferably similar, apparatus. Preferably, the further apparatus is also arranged to perform an operation based on the contextual information. This enables the sharing of contextual information directly between user devices without the need for an intermediate server.

Optionally, the apparatus is arranged to receive data signals from and/or send data signals to a server. Preferably, the server is not arranged to perform an operation based on the contextual information.

Preferably, the means for receiving data signals is adapted to receive data signals in dependence on the occurrence of an event. This enables data sharing to be performed efficiently, for example data is only shared when it has changed, or when it is determined to be of potential use.

Preferably, the event is at least one of: a change in a value relating to contextual information being detected; a value relating to contextual information exceeding; and/or a value relating to contextual information falling below a threshold value. For example, the event may be a change in mood being detected, a change in activity being detected.

Optionally, the event is at least one of: the apparatus moving into the area of a certain device; the apparatus connecting to a network; the apparatus turning on; the apparatus entering a geographic area; the user starting and/or finishing an activity; the contextual information indicating a change from a historic value and/or a baseline value, preferably a change of a predetermined magnitude.

Preferably, the event occurs externally to the apparatus. This enables the apparatus to receive contextual information in response to an event of which it might not otherwise be aware. Receipt in dependence on an external event may be used to prompt the apparatus to determine an appropriate action.

Preferably, the means for receiving data signals is adapted to receive data signals without input from the user. This enables appropriate operations to be determine and performed, or suggested, without input from the user. For example, music that suits the mood of the user may be played without the user requesting the music; this improves the user experience.

### Performing Operations

Optionally, the means for performing an operation comprises a neural network. Machine learning techniques may also or alternatively be used.

An apparatus according to any preceding claim, wherein the means for performing an operation is adapted to perform an operation in dependence on an attribute of the user, such as at least one of: the activity of the user; the mood of the user; the availability of the user; a time until the next scheduled activity of the user; the surroundings of the user; companions in the proximity of the user.

Preferably, the means for performing an operation is adapted to recommend an activity to the user.

Optionally, the means for performing an operation is adapted: to recommend audio and/or video; to allow access to an area; to operate an actuator; to operate a speaker and/or display; to operate a robot; to perform audio and/or video; to suggest advertising; and to alter a recommendation.

Preferably, the apparatus further comprises means for receiving non-contextual information.

The means for receiving non-contextual information is typically similar to or the same as the means for receiving contextual information, e.g. the means for receiving non-contextual information may be a communication interface.

Preferably, the apparatus further comprises means for determining a non-contextual operation in dependence on the non-contextual information; and means for modifying the non-contextual operation in dependence on the contextual information. This enables an operation to be determined based on, for example, past choices of the user or of other users and this operation to be modified depending on the current status of the user. As an example, a music recommendation based on listening habits of other users (e.g. the selection of a popular song) may be modified based on a user's mood at the time of the recommendation being made.

The means for performing an non-contextual operation is typically similar to or the same as the means for determining an operation, e.g. the means for determining a non-contextual operation may be a processor or circuit.

### Permissioned Sharing

Preferably, the apparatus further comprises means for accepting user input, preferably wherein the means for receiving data signals is adapted to receive data signals in dependence on input from a user, preferably permission from a user.

This ensures that information, which may be sensitive, is not shared without consent from the user.

The means for accepting user input is typically a user interface, such as a touchscreen, a keyboard, or a mouse.

Preferably, the apparatus further comprises means for determining whether the apparatus has permission to share the contextual information. This determination may comprise consideration of previous permissions granted by the user, such as consideration of permissions granted to the apparatus or other apparatuses at previous times. By determining a permission without user input, the user experience may be improved.

The means for determining whether the apparatus has permission is typically similar to or the same as the means for determining an operation, e.g. the means for determining whether the apparatus has permission may be a processor or circuit.

Preferably, the means for determining whether the apparatus has permission is dependent on at least one of: a user input; the type of contextual information being requested; the location of the apparatus; the time; a network connection status of the apparatus; a certificate held by the apparatus; the type of the apparatus; and whether the apparatus is part of a list of approved apparatuses.

Optionally, the received data signals comprise encrypted data and/or wherein the received data signals comprise data that is encrypted after receipt. This further ensures that, possibly sensitive, user information is not compromised.

Preferably, the means for receiving data received data signals comprise is adapted to receive data received data signals comprise using at least one of: Bluetooth®; Near Field Communication (NFC); infrared; an area network; a wide area network; and a local area network.

### Coordinator and Receiver

Preferably, the means for receiving data is adapted to query a connection status relating to a/the transmitting application. This enables sharing to only be commenced when the transmitting application is available and ready to share data signals.

Preferably, the apparatus further comprises means for determining a receiving application, the receiving application being arranged to receive data, and/or a means for determining a coordinating application, the coordinating application being arranged to transmit data.

The means for determining a receiving application is typically similar to or the same as the means for determining an operation, e.g. the means for determining a receiving application may be a processor or circuit.

Optionally, the means for determining a receiving application and/or a coordinating application comprises means for determining based on the time at which the apparatus initiated a data sharing session. The apparatus that has been available for sharing for a longer time may be set as the receiving application, since this application may have access to historic data that was shared before the apparatus initiated the data sharing session.

Optionally, the means for determining a receiving application and/or a coordinating application is adapted to designate an application that first initiated the data sharing session as the coordinating application.

Optionally, the means for determining a receiving application and/or a coordinating application is adapted to determine a receiving application and/or a coordinating application based on at least one of: a type of the apparatus; a type of contextual information being requested; the location of the apparatus; a time; a network connection status of the respective applications; a certificate held by one of the applications; and whether each application is on a list of approved applications.

According to a further aspect of the disclosure herein, there is provided an apparatus according to any preceding claim, further comprising: means for determining the status of a second apparatus; and means for joining a data sharing session as either a coordinating apparatus or a receiving apparatus in dependence on the status of the second apparatus; wherein: the coordinating apparatus is arranged to transmit data signals; the receiving apparatus is arranged to receive data signals; and the data signals contain contextual information relating to a user.

Optionally, the apparatus is arranged to receive and/or transmit signals using at least one of: pressure waves, sound waves, ultrasonic waves, and/or electromagnetic waves.

Optionally, the apparatus is arranged to receive and/or transmit signals using infrared and/or visible light (e.g. using Li-Fi).

The two apparatuses may comprise two applications on the same device, where sharing may occur between the two applications.

According to a further aspect of the disclosure herein, there is provided an apparatus for sharing data, the apparatus comprising: means for determining the status of a second apparatus; means for joining a data sharing session as either a coordinating apparatus or a receiving apparatus in dependence on the status of the second apparatus; wherein the coordinating apparatus is arranged to transmit data signals; wherein the receiving apparatus is arranged to receive data signals; and means for sharing data received data signals comprise with the second apparatus; wherein the data signals contain contextual information relating to a user.

Optionally, the means for joining a data sharing session comprises means for joining as a receiving apparatus when the second apparatus is available to share data received data signals comprise and/or the means for joining a data sharing session comprises means for joining as a coordinating apparatus when the second apparatus is not available to share data received data signals comprise.

Preferably, the means for determining the status of a second apparatus is adapted to determine at least one of: the availability of the second apparatus to share data; the type of device on which the second apparatus is implemented; the communication capability of the second apparatus; and a permission related to the second apparatus.

Optionally, the apparatus further comprises means for receiving a status request from a third apparatus; and means for transmitting an indication of whether the first apparatus is the coordinating apparatus and/or the receiving apparatus. This is useable to indicate whether the third apparatus should request and/or receive data from the first apparatus or the second apparatus (or any other apparatus).

Preferably, where the first apparatus is the coordinating apparatus, the means for joining a data sharing session comprises means for initiating a data sharing session

Optionally, the apparatus further comprises: means for leaving the data sharing session; and means for designating the second apparatus and/or a further apparatus as the coordinating application in dependence on the status of the first apparatus. This enables the apparatus to hand down the status of coordinating application to another apparatus as it leaves the data sharing session, so that there may be an unbroken chain of coordinating applications, and data may remain available even once the apparatus that initially shared the data has left the data sharing session.

Optionally, when the first apparatus is the coordinating application, the means for leaving the data sharing session comprises means for designating the second apparatus as the coordinating apparatus.

The apparatus may comprise at least one of: a phone; a watch; a speaker; a computer; glasses; earphones; footwear; and/or clothing. Generally, the apparatus may comprise any device that comprises a “smart” capability and/or a processor.

According to another aspect of the present disclosure, there is provided a system comprising: a first application; and a second application; wherein the first application comprises: means for receiving data signals from the second application, wherein the data signals contain contextual information relating to a user; and means for performing an operation in dependence on the contextual information; and wherein the second application comprises: means for transmitting data signals to the first application.

The first application may be located on any apparatus described herein. The second application may be located on any apparatus described herein. The second application may be located on a further apparatus external to and/or separate from the apparatus on which the first application is located.

Preferably, each application comprises a corresponding database containing contextual information, preferably wherein each database is equivalent. Each database may be updated each time that contextual information is transmitted. One or more transmissions of data may comprise transmission to the database, where receiving data may comprise receiving data from the database and/or querying the database.

According to another aspect of the present disclosure, there is provided a method of sharing data, the method comprising: receiving data signals, wherein the data signals contain contextual information relating to a user; and performing an operation in dependence on the contextual information.

An apparatus for sharing data, the apparatus comprising: means for transmitting data signals, wherein the data signals contain contextual information relating to a user; wherein the contextual information is arranged to be used within the performance of an operation.

Preferably, the means for transmitting data signals is adapted to transmit data in dependence on the occurrence of an event.

According to another aspect of the present disclosure, there is provided a method of sharing data, the method comprising: at a first application: transmitting data signals to a second application, wherein the data signals contain contextual information relating to a user; wherein the contextual information is arranged to be used within the performance of an operation at the second application.

According to another aspect of the present invention, there is disclosed an apparatus for sharing data, the apparatus comprising: means for storing contextual information relating to a user, wherein the contextual information comprises information inferred from sensor data; means for receiving a request; and means for transmitting data signals in response to the request, wherein the data signals include contextual information.

Optionally, the means for storing contextual information comprises a storage module and/or a memory module, such as a hard drive or computer memory (e.g. RAM or ROM).

Optionally, the means for transmitting data signals comprises a communications interface.

Preferably, the request comprises a request for contextual information.

According to another aspect of the present invention, there is disclosed an apparatus for sharing data, the apparatus comprising: means for receiving sensor data from one or more devices; means for inferring contextual information relating to a user, wherein the contextual information comprises information inferred from the sensor data; means for receiving a request; and means for transmitting data signals in response to the request, wherein the data signals include contextual information.

Optionally, the means for receiving sensor data comprises a communications interface and/or a sensor module, where the sensor module may be arranged to communicate with one or more sensors of the apparatus. Optionally, the means for inferring contextual information comprises a processor. Optionally, the means for receiving a request comprises a communications interface. Optionally, the means for transmitting data signals comprises a communications interface.

Preferably, the data signals comprise a plurality of different types of contextual information.

Preferably, the apparatus further comprises means for determining a permission of a device receiving the data signals before transmitting the data signals. Preferably, the apparatus is arranged to transmit the data signals only if the receiving device has a required permission.

Optionally, the means for determining a permission comprises a processor.

According to another aspect of the present invention, there is disclosed a method of sharing data, the method comprising: storing contextual information relating to a user, wherein the contextual information comprises information inferred from sensor data; receiving a request; and transmitting data signals in response to the request, wherein the data signals include contextual information.

According to another aspect of the present invention, there is disclosed a method of sharing data, the apparatus comprising: receiving sensor data from one or more devices; inferring contextual information relating to a user, wherein the contextual information comprises information inferred from the sensor data; receiving a request; and transmitting data signals in response to the request, wherein the data signals include contextual information.

More generally, there is described a method of using any apparatus as described herein.

In general, a method and apparatus are disclosed herein that are suitable for the sharing of data. This data comprises contextual information, which is preferably information that is inferred from other information. The contextual information is used to perform an operation, for example the operation may be the performance of audio and/or video.

The operation may be the operation of an actuator. The method and/or apparatus may include one or more of the following features:
Inferring contextual information from information, for example inferring contextual information from raw data and/or sensor data.
Inferring contextual information from a plurality of sources, preferably where the sources comprise different sensors, different applications and/or different devices. The sensors, application and devices may each be separate from the inferring device.
Sharing information that is not directly related to the performance of the operation.
Sharing non-contextual information and/or information that is directly related to the performance of the operation, preferably combining contextual and non-contextual information.
Sharing information in dependence on the operation to be performed, preferably in dependence on the type of information.
Sharing information in dependence on an event, preferably transmitting information in dependence upon an event.
Updating and/or querying a database containing contextual information.
Sharing information, e.g. between devices, at time intervals, preferably wherein the time intervals depend on the contextual information being shared.
Receiving contextual information from and/or transmitting contextual information to a, preferably separate, device.
Transmitting contextual information to a device that is not capable of otherwise determining the contextual information.
Receiving contextual information in dependence on one or more permissions, preferably where the permissions are configured by a user and wherein the permissions relate to permissions for sharing data with a device and/or for sharing a type of contextual information.
Determining a contextual history based on contextual information;
Determining a baseline value relating to contextual information, preferably where the baseline value depends on the user.
Determining a variance of a value from a baseline value and/or a historical value, preferably sharing information in dependence on the variation.
Sharing information dependent on an input from a user.
Sharing information without input from a user.
Sharing information dependent on a configuration set up by a user prior to the sharing.
Performing an operation dependant on contextual information using an actor, a robotic component, an actuator, a display and/or a speaker.
Configuring a permission relating to the sharing of information, preferably relating to the sharing of a type of contextual information and/or the sharing of information with a device.
Determining the behaviour of a user in a context.
Using previously determined behaviours in the determination of operations.

Further described herein is a method of learning which operations a user performs in certain contexts. Performing operations inferred from contextual information may be difficult before learning from experience what operations are relevant to certain situations. For example, it is fine to know that a user is currently stressed, but that information alone is not enough to know what music to play. However, if it is known what music users listen to when stressed, it can be learned what music they enjoy in this context, and this music can be suggested (or music that has in some other way been deemed to be similar, e.g. same artist or genre) when the user is next found to be stressed.

It can also be appreciated that the methods can be implemented, at least in part, using computer program code. According to another aspect of the present disclosure, there is therefore provided computer software or computer program code adapted to carry out these methods described above when processed by a computer processing means. The computer software or computer program code can be carried by computer readable medium, and in particular a non-transitory computer readable medium. The medium may be a physical storage medium such as a Read Only Memory (ROM) chip or a Hard Disk Drive (HDD). Alternatively, it may be a disk such as a Digital Video Disk (DVD-ROM) or Compact Disk (CD-ROM). It could also be a signal such as an electronic signal over wires, an optical signal or a radio signal such as to a satellite or the like. The disclosure also extends to a processor running the software or code, e.g. a computer configured to carry out the methods described above.

Any feature described as being carried out by an apparatus, an application, and a device may be carried out by any of an apparatus, an application, or a device. Where multiple apparatuses are described, each apparatus may be located on a single device.

Any feature in one aspect of the disclosure may be applied to other aspects of the invention, in any appropriate combination. In particular, method aspects may be applied to apparatus aspects, and vice versa.

Furthermore, features implemented in hardware may be implemented in software, and vice versa. Any reference to software and hardware features herein should be construed accordingly.

Any apparatus feature as described herein may also be provided as a method feature, and vice versa. As used herein, means plus function features may be expressed alternatively in terms of their corresponding structure, such as a suitably programmed processor and associated memory.

It should also be appreciated that particular combinations of the various features described and defined in any aspects of the disclosure can be implemented and/or supplied and/or used independently.

The disclosure also provides a computer program and a computer program product comprising software code adapted, when executed on a data processing apparatus, to perform any of the methods described herein, including any or all of their component steps.

The disclosure also provides a computer program and a computer program product comprising software code which, when executed on a data processing apparatus, comprises any of the apparatus features described herein.

The disclosure also provides a computer program and a computer program product having an operating system which supports a computer program for carrying out any of the methods described herein and/or for embodying any of the apparatus features described herein.

The disclosure also provides a computer readable medium having stored thereon the computer program as aforesaid.

The disclosure also provides a signal carrying the computer program as aforesaid, and a method of transmitting such a signal.

The disclosure extends to methods and/or apparatus substantially as herein described with reference to the accompanying drawings.

The disclosure will now be described, by way of example, with reference to the accompanying drawings.

### DESCRIPTION OF THE DRAWINGS

FIG. 1 shows a system containing a user and multiple devices;

FIG. 2 is an illustration of a generic computer device;

FIG. 3 is an illustration of a control panel relating to contextual information;

FIG. 4 is an illustration of a control panel relating to devices;

FIG. 5 shows a database for storing contextual information;

FIG. 6 is a flowchart of a method of determining contextual information;

FIG. 7 is a flowchart of a method of sharing information;

FIG. 8 is a flowchart of a method of sharing information based on restrictive permissions;

FIG. 9 is a flowchart of a coordinator-based method of sharing information; and

FIG. 10 shows a method of performing operations based on contextual information.

### DESCRIPTION OF THE PREFERRED EMBODIMENTS

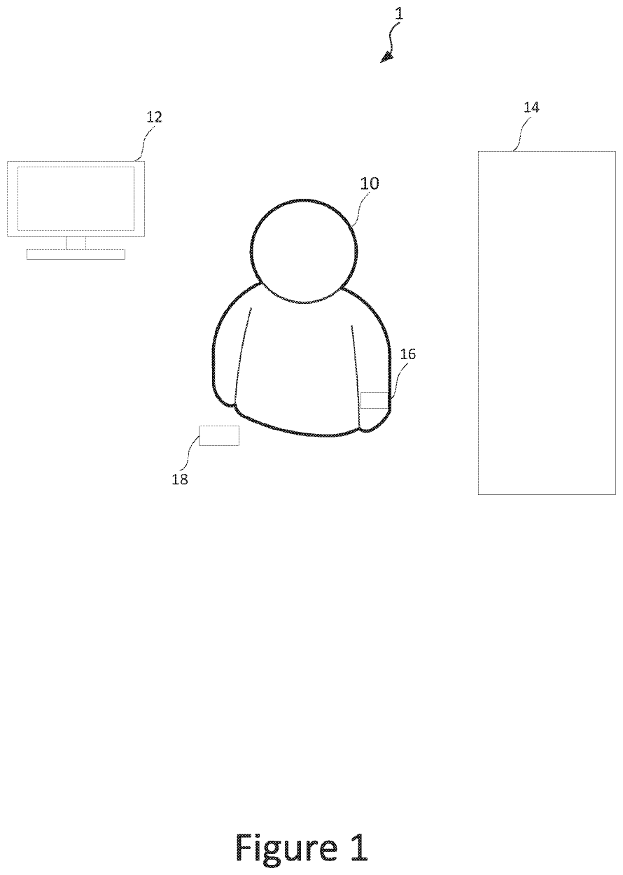

Referring to FIG. 1, an exemplary system 1 comprises a user 10 and a number of digital devices 12, 14, 16, 18. These devices comprise both ambient devices, which are normally fixed in place, and personal devices, which are normally portable. In the exemplary system 1, ambient devices include a television 12 and a fridge 14 and portable devices include a watch 16 and a phone 18.

The ambient devices are typically not moved on a regular basis and are thus are only in proximity to the user 10 when the user 10 is in a certain location. The personal devices are typically regularly moved and are thus in proximity to the user 10 while the user 10 moves between locations. The television 12 and the fridge 14 are only in proximity to the user 10 when the user 10 is at their home; the user 10 takes the watch 16 and the phone 18 when the user 10 leaves their home.

The devices 12, 14, 16, 18 are each adapted to obtain information relating to the user 10. As examples, the television 12 collects information about the viewing schedule and preferences of the user 10, and the watch 16 collects information about the heart rate and routine of the user 10.

Further the devices 12, 14, 16, 18 are each adapted to communicate data, so that each device can receive and/or transmit data to at least one other device. In some embodiments, the devices 12, 14, 16, 18 are configured to use Bluetooth® to communicate data with each other, e.g. to share data with each other.

The present disclosure relates generally to a method of and apparatus for a first device communicating with a second device to share information relating to the user. In particular, the present disclosure relates to the devices sharing contextual information. Additionally, the present disclosure relates to a method of devices sharing contextual information to enable appropriate operations to be carried out while minimising the input required from the user 10.

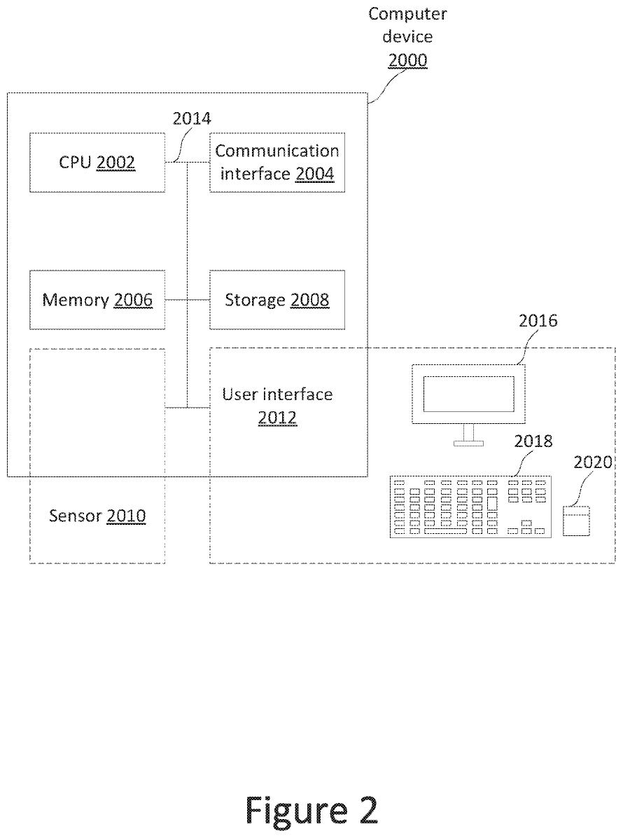

Referring to FIG. 2, each of the television 12, the fridge 14, the watch 16, and the phone 18 contains a respective computer device 2000. Each computer device 2000 comprises a processor in the form of a CPU 2002, a communication interface 2004, a memory 2006, storage 2008, a sensor 2010 and a user interface 2012 coupled to one another by a bus 2014. The user interface 2012 comprises a display 2016 and an input/output device, which in this embodiment is a keyboard 2018 and a mouse 2020.

The CPU 2002 is a computer processor, e.g. a microprocessor. It is arranged to execute instructions in the form of computer executable code, including instructions stored in the memory 2006 and the storage 2008. The instructions executed by the CPU 2002 include instructions for coordinating operation of the other components of the computer device 2000, such as instructions for controlling the communication interface 2004 as well as other features of a computer device 2000 such as a user interface 2012. In various embodiments, the CPU comprises a field programmable gate array (FPGA), coarse grain reconfigurable array (CGRA), or an application specific integrated circuit (ASIC). It will be appreciated that a variety of other circuits, e.g. logic circuits, and electrical components may be used to perform operations and/or implement methods using software or hardware.

The memory 2006 is implemented as one or more memory units providing Random Access Memory (RAM) for the computer device 2000. In the illustrated embodiment, the memory 2006 is a volatile memory, for example in the form of an on-chip RAM integrated with the CPU 2002 using System-on-Chip (SoC) architecture. However, in other embodiments, the memory 2006 is separate from the CPU 2002. The memory 2006 is arranged to store the instructions processed by the CPU 2002, in the form of computer executable code. Typically, only selected elements of the computer executable code are stored by the memory 2006 at any one time, which selected elements define the instructions essential to the operations of the computer device 2000 being carried out at the particular time. In other words, the computer executable code is stored transiently in the memory 2006 whilst some particular process is handled by the CPU 2002.

The storage 2008 is provided integral to and/or removable from the computer device 2000, in the form of a non-volatile memory. The storage 2008 is in most embodiments embedded on the same chip as the CPU 2002 and the memory 2006, using SoC architecture, e.g. by being implemented as a Multiple-Time Programmable (MTP) array. However, in other embodiments, the storage 2008 is an embedded or external flash memory, or such like. The storage 2008 stores computer executable code defining the instructions processed by the CPU 2002. The storage 2008 stores the computer executable code permanently or semi permanently, e.g. until overwritten. That is, the computer executable code is stored in the storage 2008 non-transiently. Typically, the computer executable code stored by the storage 2008 relates to instructions fundamental to the operation of the CPU 2002.

The communication interface 2004 is configured to support short-range wireless communication, in particular Bluetooth® and Wi-Fi communication, long-range wireless communication, in particular cellular communication, and an Ethernet network adaptor. In particular, the communications interface 2004 is configured to establish communication connections with other computing devices. Depending on the device the computer device 2000 is associated with, some or all of the communication methods are used.

As will be appreciated by the skilled person, the sharing of contextual information may be medium agnostic, where any means of communication may be used to share contextual information. In particular, the contextual information may be communicated using any form of wave. Therefore, the communication interface 2004 may be configured to support communication via pressure waves, sound waves, ultrasonic waves, and/or electromagnetic waves. As an example, contextual information may be encoded in the sound waves produced by a speaker, in order to enable the speaker to share contextual information.

Similarly, the communication interface 2004 may be configured to communicate via infrared or visible light (e.g. using Li-Fi); this is useful where smart lightbulbs communicate contextual information to ambient devices.

The storage 2008 provides mass storage for the computer device 2000. In different implementations, the storage 2008 is an integral storage device in the form of a hard disk device, a flash memory or some other similar solid state memory device, or an array of such devices.

The sensor 2010 is configured to obtain information relating to the device 2000, the user 10, and/or the environment. In different implementations, the sensor 2010 is a GPS sensor, a temperature sensor, a proximity sensor, a heart rate sensor, an accelerometer, an infrared sensor, an impact sensor, a pressure sensor (e.g. a barometer), and/or a gyroscope. It will be appreciated that a number of other sensors as are known in the art may also be used. Typically, the computer device 2000 contains a plurality of sensors, where the sensors are adapted to provide sensory data to an application on the computer device 2000.

In some embodiments, there is provided removable storage, which provides auxiliary storage for the computer device 2000. In different implementations, the removable storage is a storage medium for a removable storage device, such as an optical disk, for example a Digital Versatile Disk (DVD), a portable flash drive or some other similar portable solid state memory device, or an array of such devices. In other embodiments, the removable storage is remote from the computer device 2000, and comprises a network storage device or a cloud-based storage device.

The computer devices 2000 contained by the television 12, the fridge 14, the watch 16 and the phone 18 may be the same, but in most implementations the computer devices 2000 will differ from one another somewhat to suit the different specific purposes and functions of the television 12, the fridge 14, the watch 16 and the phone 18 respectively. For example, the primary function of the television 12 is to display video. The display 2016 of the computer device 2000 on which the television 12 is implemented therefore typically is large. The watch 16 and the phone 18 are portable; the computer devices 2000 on which the watch 16 and the phone are implemented therefore typically have a compact user interface 2012, which may comprise a touchscreen.

The user interface 2012 typically comprises one or more actors that are adapted to perform operations that have an effect external to the computer device 2000. In some embodiments, this is the display 2016, which is useable to display information and/or video. In some embodiments, the user interface 2012 comprises a speaker, a locking mechanism, an actuator and/or a robot. The user interface 2012 is in various embodiments, adapted to: play music and/or audio, lock and/or unlock a door or other access means; grasp; move; interact with an object; and interact with the user 10.

A computer program product is provided that includes instructions for carrying out aspects of the method(s) described below. The computer program product is stored, at different stages, in any one of the memory 2006, storage device 2008 and removable storage. The storage of the computer program product is non-transitory, except when instructions included in the computer program product are being executed by the CPU 2002, in which case the instructions are sometimes stored temporarily in the CPU 2002 or memory 2006. It should also be noted that the removable storage 2008 is removable from the computer device 2000, such that the computer program product is held separately from the computer device 2000 from time to time.

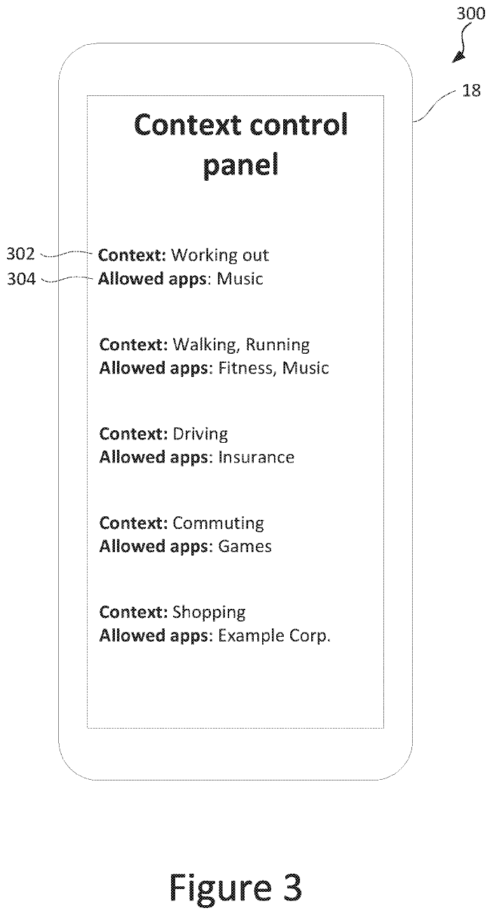

Referring to FIG. 3, there is shown the user interface 2012 of the phone 18 being used to display a context control panel 300.

The context control panel 300 is adapted to enable the user 10 to view or alter settings relating to the sharing of data. Typically, this comprises the user 10 being able to view or alter the applications or devices that are allowed to share data for a given context and/or that are able to share a certain type of contextual information. In this embodiment, the context control panel 300 displays a context 302 along with an indication of the allowed applications 304 that are allowed to share data for that context.

The user 10 is able to define a context, or select a context from a pre-set list, and then add applications that are allowed to share data for this context. Being allowed to share data comprises being allowed to transmit data and/or being allowed to receive data.

The context of the user is determined by one or more devices, e.g. the phone 18 is able to determine an expected future location based on previous user movements, the watch 16 is able to determine a stress level based upon a heart rate.

Contextual information, e.g. information relating to the context of the user, is useable to personalise a user experience; it can be used to determine the desires, intentions, and mood of the user 10 so that suitable operations can be performed with minimal input from the user 10. Typically, suitable operations depend on the present context of the user, e.g. whether the user is stressed or busy. The sharing of data between devices enables each device to determine this context.

The operations that are performable using contextual information differ from those performable using raw sensor data; as an example, raw input from an accelerometer is sometimes used to change a display from a landscape mode to a portrait mode. The present disclosure envisages further usages of this raw sensor data, for example, by way of making inferences based on the raw accelerometer data (alongside other data—e.g. time data) the phone 18 is able to determine that the change in orientation is due to the user 10 placing the phone 18 on a table in a bedroom late at night (and so it is likely that the user 10 is going to bed).

Exemplary context types are shown in the table below

| Context Type | Examples |
|---|---|
| Activity | Stationary, Walking, Running, Cycling, |
| | Bicycle, Bus, Car, Motorbike, Subway, |
| | Train, Tram, Boat |
| Indoor/Outdoor | Indoor, Outdoor, Enclosed |
| Place | Home, Work, Academic, Entertainment, |
| | Food & Drink, Office, Recreational, |
| | Residential, Shops & Services |
| Situation | Housework, Leisure, Morning Rituals, |
| | Shopping, Sleeping, Social, Travelling, |
| | Working, Working Out |
| (Relative) Heart Rate | Resting, Elevated, Maximum |
| Heart Rate Variability | Low, Medium, High |
| People Presence | Yes/No, Number of people nearby |
| Stress | Low, Medium, High. Quantitative values |
| Mood/state of mind | Valence, Arousal, Dominance |

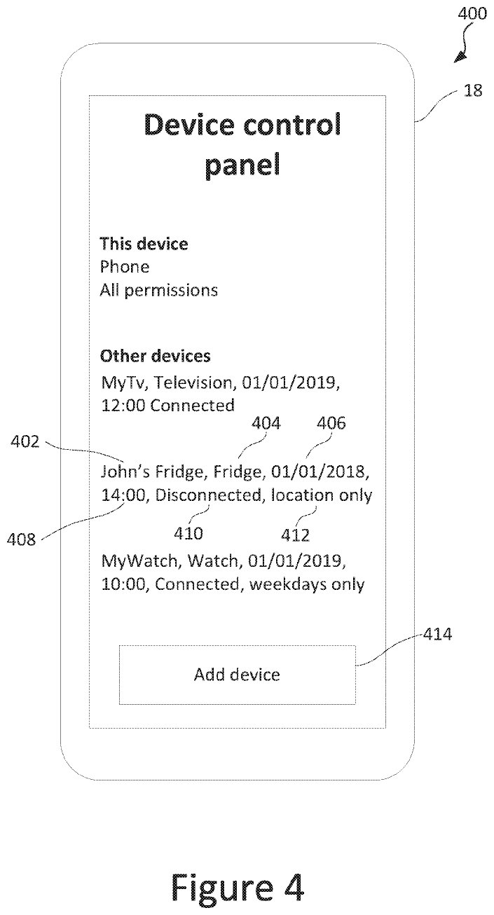

Referring to FIG. 4, there is shown the user interface 2012 of the phone 18 being used to display a device control panel 400.

The device control panel 400 is adapted to enable the user 10 to control the permissions of devices and view details of the operation of devices.

The device control panel 400 displays a device name 402, a device type 404, a date of last connection 306, a time of last connection 410, a connection status 410, and permission information 412.

The device control panel 400 also comprises an “add device” functionality 414, which in this embodiment is a button. Adding a device typically comprises using the communication interface 2004 to search for devices that are nearby or that are using the same network, for example devices on a shared Bluetooth®, WiFi network, or other mesh network may be detected and the user 10 may be asked whether these devices should be added. Devices may also be added based on an IP address, a name, or another identifier.

The permission information 412 relates to the type of sharing that is allowed for a device. This relates to: the types of information that can be shared; the timeframes during which information can be shared; the distance at which a device is allowed to share information; the group of devices with which sharing is allowed; and activities during which sharing is allowed. This permission information 412 generally also contains a subset of the information from the context control panel 300. It will be appreciated that other permissions may also be considered.

The permission information 412 also relates to the extent to which storage and analysis of information is allowed for each device. By storing and analysing information on only certain devices, all personal information remains on trusted devices. This allows users to enable sharing with public devices (e.g. train ticket barriers) without fear that their personal information will be compromised, since the entirety of the information shared is controlled and visible.

In various embodiments, the contextual information is natively stored on the device in at least one of: a database, text file, binary file, device settings, and an application space.

To prevent sensitive information from being exposed in the case of device theft, in some embodiments the contextual information is encrypted and made accessible only after authentication (e.g. using facial recognition, fingerprints, or audio recognition).

In some embodiments, a device can be added based on a request from that device; for example, a ticket barrier at a train station may be configured to search for phones using Bluetooth® and request the sharing of data from these devices.

This request results in the user 10 being shown a prompt on the phone 18 querying whether data sharing with the barrier is permitted. The user can then accept or reject the request.

In some embodiments, only certain devices are able to request addition to the device list, these certain devices may be those devices owned by the user, or those devices having certain certificates installed. This prevents the user 10 from being irritated by unwanted prompts from devices.

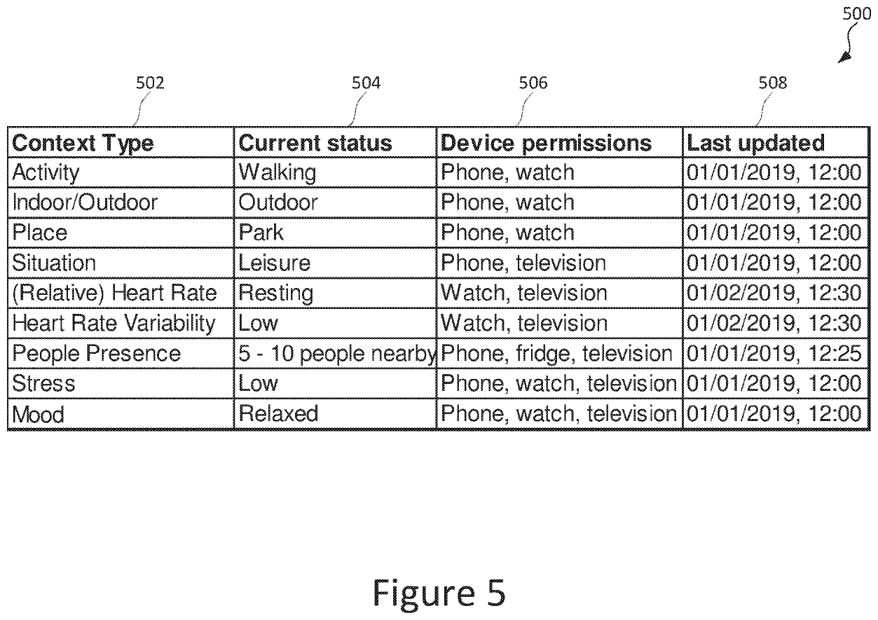

Referring to FIG. 5, there is shown a database 500 that is useable for storing contextual information.

The database 500 comprises information relating to: a context type 502; a current status 504 for each context type; device permissions 506 for each context type; and a time of last update 508 for each context type.

The context type 502 relates to the types of information available. Exemplary context types have been described with reference to FIG. 3.

The current status 504 relates to an assessment of the present situation of the user 10 for the related context type. The device permissions 506 relate to which devices are capable of accessing information relating to each context type. The time of last update 508 relates to the time and date at which the information for each context type was last updated.

Typically, copies of the database 500 are stored on one or more devices. For each context type 502, information relating to that context type in the database 500 is configured to be updatable by any device with permission to modify information relating to that context type.

For each context type 502, information relating to that context type in the database 500 is configured to be readable by any device with permission to read information relating to that context type. Typically, devices which are able to read information relating to a context type are also able to modify information relating to a context type; however, in some embodiments there are separate permissions for whether each device is able to read, modify, analyse, or execute the information stored in the database 500 for each context type 502.

The copies of the database 500 on each device are updated either at set time intervals, which may differ for each device and each context type 502, or upon the occurrence of an event. As an example, the database 500 on the phone 18 is typically updated when the phone 18 connects to a network.

In some embodiments, the database 500 is updated when one or more of the devices detect a change in contextual information, for example when the mood of a user changes, or when a stress measure changes from “not stressed” to “stressed”. This change may trigger the updating of the database and/or the transmission of data. Typically, the database 500 is updated when a significance threshold is exceeded, where this may relate to a value changing by a certain magnitude; changing by a certain percentage; and/or exceeding or passing below a threshold value. By storing information in the database 500 and updating the database 500 regularly, data is transferable between devices regardless of the connection status of each device at the time of data sharing. As an example, the watch 16 is adapted to determine the heart rate variability regularly and update the database 500 accordingly, the television 12 is thus capable of accessing heart rate variability information even when the watch 16 has run out of battery.

Typically, equivalent databases are stored on each device and each database is encrypted so that devices are only capable of accessing data for context types specified by the user 10. As an example, although information relating to the activity of the user 10 is stored on the television 12, the television 12 is not capable of accessing this information.

In some embodiments, the database stored on each device depends upon that device, for example in some embodiments each device comprises a database containing only contextual information which that device has permission to access. Each device may then have a personal (and unique) database, or databases may be shared for a number of devices having equivalent permissions. In some embodiments, devices may be grouped by permissions, so that a first group of devices has a first set of permissions and a second group of devices has a second set of permissions.

Each device may have permission, e.g. be authorised, to access, receive, or infer, only certain types of contextual information, where the combination of different types of contextual information enables the performance of specific operations. One or more permissions of a device may be stored in a database and/or provided to a sharing device at the time of sharing contextual information or sensor data.

Sensor data may be useable by a device for only certain inferences of contextual information. With an exemplary device, heart beat data is useable to infer a stress level, but not an activity—therefore the exemplary device may be arranged to receive heart beat data continuously, but to only be able to use this data for certain purposes.

Similarly, this exemplary device may be arranged (e.g. permitted) to receive a (inferred) stress level from another device and/or server, where this stress level is inferred from heart beat data, but the exemplary device may be arranged to not be able to receive an activity inferred from heart beat data.

In some embodiments, the exemplary device may be arranged (e.g. permitted) to receive contextual information relating to an activity, where this contextual information is based on heart beat data. This may be possible even where the exemplary device is not permitted to receive heart beat data and/or use heart beat data to infer an activity. This enables sharing of non-sensitive contextual information where the sensitive sensor data used to infer this information is not shared.

Each of these options is typically selected by the user; this selection may comprise the user assigning a sensitivity level to sensor data and/or types of contextual information and then recording for each device an access level that indicates which sensitivity level the device is able to access.

This control of permissions for receiving/sharing/inferring contextual information enables fine tuning of personalised operations, while allowing the user to retain close control over the use of their data.

This also enables closer control over the data of the user than, for example, the sharing of a singular context. The use of multiple different types of contextual information, which may be inferred using data from various sensors, allows certain sensors to be used to infer certain contextual information as desired, and this contextual information to only be disseminated as desired.

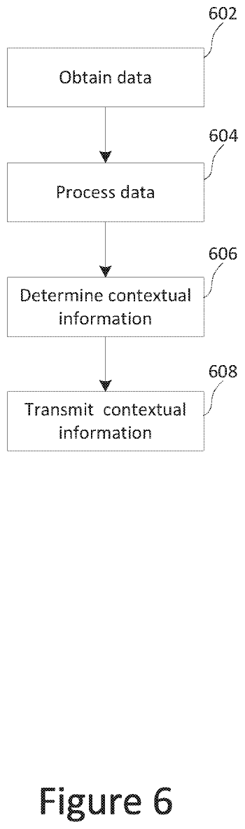

Referring to FIG. 6, there is shown an exemplary method of determining contextual information from raw sensor data.

In a first step 602, a device obtains data. This typically comprises obtaining data from the sensor 2010 of the device. In some embodiments, this comprises obtaining data via the communication interface 2004 of the device, where this allows the device to obtain data that it is not able to obtain using the sensor 2010. As an example, the television does not comprise a temperature sensor; yet the television is capable of receiving temperature information via a Bluetooth® connection with the phone 18.

Types of raw data which are not physical measurements taken by a sensor may be obtained, for example calendar data.

In a second step 604, the data is processed. Processing comprises performing analysis on received (raw) data and/or modifying the format of received (raw) data.

In a third step 606, contextual information is determined based on the processed data, typically contextual information is inferred from the processed data. As an example, an indication of stress is obtainable from raw heart rate data, and an indication of activity is obtainable from location data, time data, and data regarding nearby devices. The phone 18 being in a restaurant during the evening is useable to infer the user is at dinner; data relating to nearby devices is useable to determine dining companions. Contextual information is useable to determine appropriate actions based on, for example, the user's mood that are not determinable using raw data.

In another example, the heart rate of the user as detected, for example, by the watch 16 and the motion of the user, as detected for example by the phone 18, are used to infer a stress level; a heart rate that is greater than a baseline heart rate, e.g. a heart rate of 150 beats per minute for a user that has a resting heart rate of 120 beats per minute indicates an elevation in heart rate. If the motion of the user is detected as rapid, the user is determined as exercising, if the motion of the user is sedentary, the user is determined as stressed and/or angry.

Contextual information is determined based on at least one type of obtained raw data; typically contextual information is determined based on a number of types of obtained raw data, where these types of raw data may be obtained from different devices. In general, a number of data, which may be raw data, processed data and/or sensor data may be used within a determination of contextual information. The data may come from different sources, sensors and/or devices so that data may be received at a device from a number of sources and combined to determine contextual information that is not determinable using any single device. In a fourth step 608, the contextual information is transmitted in the form of data being transmitted. This transmission is typically to another device. In some embodiments, the transmission comprises updating a database, as described with reference to FIG. 5, where this database is then accessible from other devices.

In various embodiments, the determination of contextual information involves using at least one of: a neural network; boosted decision trees; markov models; recurrent neural networks; and autoencoders. In some embodiments, determining contextual information comprises the use of databases or formulae that enable contextual information values to be determined from raw data values.

Typically, contextual information either is inferred directly from raw data or is inferred indirectly from raw data. That is, contextual information may be inferred from data that is itself inferred; contextual information may also be inferred from other contextual information.

The contextual information is shared over a period of time and is used to form a history of the behaviour of the user 10. The information is stored, refreshed, shared or deleted as the user 10 sees fit, which allows the user 10 to always remain in control of their personal data. Over time the context history can be used to detect behavioural patterns (e.g. commute times, workout routines, shopping habits) to enable personalised digital experiences.

Typically, the context history comprises a measure of the previous behaviour of the user 10 in relation to a context; the context history may comprise a list of the actions previously performed by the user 10 for a certain mood, e.g. when they have been feeling stressed. As an example, the user 10 may tend to put on certain music when they are stressed; this can be detected and thereafter predicted, e.g. the device can start to play similar music once a threshold stress level is detected.

In various embodiments, the context history is formed automatically, e.g. the user 10 being stressed is determined using sensor data and their actions are then recorded, and/or using user input, e.g. the user 10 makes a list of music that they like to listen to when stressed.

The context history is continuously updated, so that user feedback is considered. If an operation is performed and received negatively, this is recorded, e.g. if the user changes a music suggestion made in response to a stress threshold being exceeded, this is taken into account for the next time that threshold is exceeded.

In some embodiments, the context history is formed based on user data obtained from public and/or private sources, for example social media data may be used to determine the user's mood at previous times and this may be compared with, e.g. music that was played at that time.

In some embodiments, the context history detects a regular routine and a baseline mood for the user 10 based on previously shared contextual information. Each device is then able to compare present contextual information to the context history to determine the present situation, mood, and activities of the user—and whether these vary from the regular/baseline values.

In some embodiments, variance of a type of contextual information from the baseline value is an event that initiates a request to communicate data.

In typical embodiments, contextual information is inferred on a first device using data on the first device and it is this contextual information that is thereafter shared with other devices. Contextual information is thus shared between devices without sharing the data used to determine the contextual information. This allows, for example, sensitive sensor data to be stored only on the first device, while (potentially non-sensitive) contextual information based on the sensitive data is shared.

In some embodiments, the contextual information also remains on a single device and is not shared across devices. The sharing of any contextual information between any device or applications depends on permissions granted by the user 10. Contextual information may be transmitted to only a limited set of devices, or it may be stored on only a limited set of devices. Certain devices may be able to request or access contextual information, but not able to store the contextual information (so, for example, access may only be permitted during a certain period after which information is deleted, or after which access is blocked).

The amount of contextual information shared between any two devices depends on the permissions, the device types, the capabilities of each device, the current context, how recent the contextual information is, the possibility of communication (e.g. whether there is WiFi available), device conditions (e.g. disable transfers in low-power and low-signal conditions), and the user's privacy settings.

In some embodiments, the user 10 is able to control the frequency with which the data sharing occurs so as to minimize the impact on battery life on both devices, especially when the applications are running in the background. This setting can be part of the context control panel 300 shown in FIG. 3.

In some embodiments, sharing occurs at set intervals; appropriate intervals can be set by the user 10 or can be determined based on the context history.

In some embodiments, sharing occurs on the occurrence of an event, such as a user entering a certain area, a device being moved into proximity of another device, or a user's heart rate exceeding a certain threshold.

Sharing based on the occurrence of events enables efficient sharing. If two or more devices are simultaneously worn or used and remain in proximity for an extended period of time, they may not need to be in continuous connection; by sharing information only when there have been significant changes the battery of each device is conserved.

While the method has been described with reference to analysis of raw data, determination of contextual information may additionally or alternatively include analysis of already processed data, of other contextual data, or analysis of a variety of different data types.

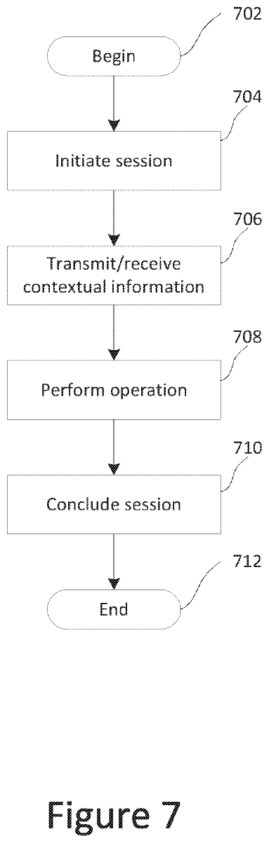

Referring to FIG. 7, there is shown an exemplary method of a device sharing data.

In a first step 702, the data sharing process begins. This is typically initiated automatically, e.g. without user input. In this example, beginning the data sharing process comprises determining the location of the user and beginning the data sharing process when the user 10 enters a certain area. The user 10 entering the area is detectable based on GPS and/or on proximity to user devices; the television 12 detects the user 10 entering a living room based on the proximity of the watch 16.

In some embodiments, the beginning of the data sharing process depends on user input. The data sharing process may be initiated by the user (e.g. the user opening an app, or pressing a “sync” button). This may involve the user being shown a prompt (e.g. “do you want to share data”), where the prompt may be generated automatically, for example based on an event.

In a second step 704, the device initiates a data sharing session. Initiation typically comprises attempting to connect to at least one other device to enable the sharing of data.

In a third step 706, the device transmits/receives data. Typically this comprises receiving/transmitting data from another connected device. The transmitted data contains contextual information relating to the user 10. The contextual information transmitted depends on the context of the user, e.g. for example the television 12 can be adapted to begin the sharing process at a certain time and thereafter request data from the phone 18. If the user 10 is on the way home, the phone 18 transmits an estimated arrival time; if the user is following directions to a restaurant, the phone 18 instead transmits an indicator that the user will return home some time later having already eaten.

Typically, the contextual information shared comprises information that is indirectly related to an operation to be performed. As an example, the information may relate to a mood that affects the type of music the user 10 would like to listen to, but this mood is not directly related to a type of music (unlike, e.g. a listening history).

In some embodiments, the method comprises communicating both contextual information and non-contextual information and combining the types of information. This enables a recommendation to be made based on the non-contextual information (e.g. music to be recommended based on a listening history) and this recommendation to be further personalised using contextual information (e.g. a subset of the recommended music to be suggested based on the mood of the user 10). Such information may be communicated at different times. In some embodiments, the shared contextual information is combined with contextual information on the receiving device.

In some embodiments, the third step 706 comprises querying a database, such as the database 500 described with reference to FIG. 5. In these instances, transmitting/receiving information may comprise transmitting/receiving information from another application or location on the same device—these embodiments still typically comprise indirectly transmitting/receiving information to/from another device.

In a fourth step 708, the device performs an operation based on the shared contextual information. In the example of the television 12 receiving data from the phone 18, the television downloads a program if the user is on their way home, whereas if the user 10 is not returning home for some time the television 12 instead enters a low-power sleep mode.

This sharing enables devices to personalise their operation using contextual information that would not otherwise be accessible. The television 12 is not able to determine the location of the user 10 by itself; however, by receiving contextual information relating to a location from the phone 18, the television is able to perform operations that benefit the user 10, such as having a program downloaded and ready to watch, without requiring input from the user 10.

In a fifth step 710, that may occur before or after the fourth step 708, the device concludes the session. This typically comprises recording the information that has been shared and details of the information sharing. These details are useable to refine operation, such as optimising sharing times and indicating which contexts it is useful to share, and to build a context history for the user 10.

In a sixth step 712, the data sharing process ends. The end of sharing enables the device to save battery by disabling the communication interface 2004.

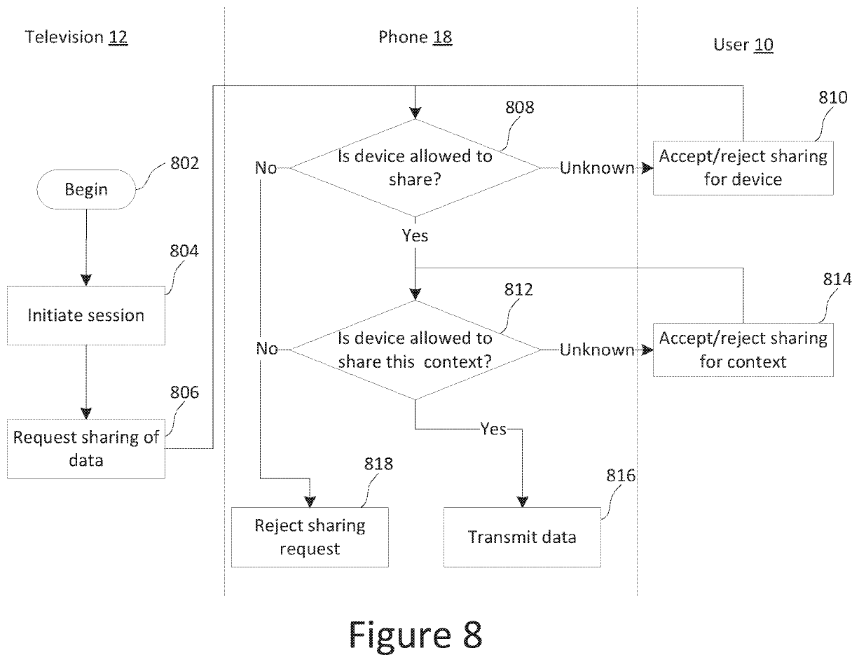

Referring to FIG. 8, there is shown a detailed method of a device requesting the sharing of data and receiving data. The two devices considered here are the television 12 and the phone 18—it will be appreciated that this method is equally applicable between any two devices.

In a first step 802, the television 12 begins the data sharing process as is described with reference to the first step 702 of FIG. 7. In a second step 804, the television 12 initiates a data sharing session as is described with reference to the second step 704 of FIG. 7—in this example the television 12 connects to the phone 18.

In a third step 806, the television 12 requests the sharing of data 806. Typically, this comprises requesting information relating to a certain context.

In a fourth step 808, the phone 18 determines whether the television 12 is allowed to share data with the phone 18.

If the television 12 has not previously been configured in the device control panel 400, in a fifth step 810 the user 10 is prompted to accept or reject the sharing request for the device.

If the television 12 is configured as not able to share data, or if the user 10 rejects the sharing request, in a ninth step 818, the sharing request is rejected.

If the television 12 is configured as able to share data, then in a sixth step 812 the phone 18 determines whether the television is allowed to share data for the present context. This comprises determining the type of contextual information that is being requested by the television 12 and determining, using the context control panel 300 whether the television 12 is able to share information for this context.

If the television 12 has not previously been configured in the context control panel 300, in a seventh step 814 the user 10 is prompted to accept or reject the sharing request for the context.

If the television 12 is configured as not able to share information for the requested context, or if the user 10 rejects the sharing request, in the ninth step 818, the sharing request is rejected.

If the television 12 is configured as able to share information for the requested context, then in an eighth step the phone 18 transmits data to the television 12.

In some embodiments, there is stored on one or more devices a database of information, as has been described with reference to FIG. 5. In these embodiments, requesting sharing of data, as has been described in reference to the third step 806, typically comprises querying the database 500 to obtain contextual information. The sixth step 812 comprises querying the database 500 to determine whether the device requesting the sharing of contextual information is able to share this contextual information. It will be appreciated that this sharing could take place on only a single device, where the database 500 may be updated from another device before the first device has begun the sharing process.

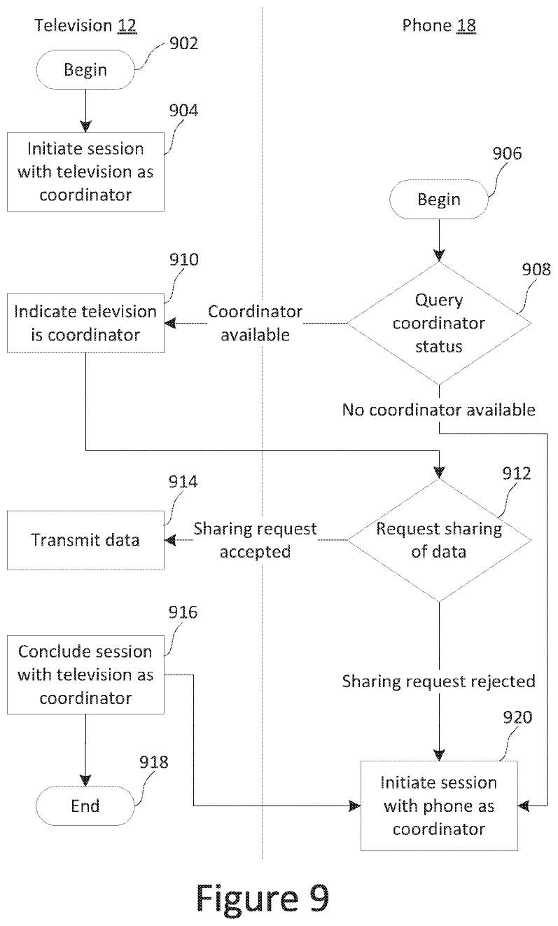

Referring to FIG. 9, there is shown a detailed exemplary method of two devices sharing data. The two devices considered here are the television 12 and the phone 18—it will be appreciated that this method is equally applicable between any two devices.

In a first step 902, the television 12 begins the data sharing process as is described with reference to the first step 702 of FIG. 7.

In a second step 904, the television 12 initiates a session as is described with reference to the second step 704 of FIG. 7. The television 12 initiates the session as a coordinator. The coordinator is the device that is adapted to share data; the decision of which device is the coordinator typically depends on the context being shared and the information available to the device.

At a time that may be before, simultaneous with, or after the time at which the first step 902 or the second step 904 occurs, in a third step 906, the phone 18 begins the data sharing process. As has been described with reference to the first step 702 of FIG. 7, the phone 18 typically begins the sharing process based on a detected proximity to the television 12. In various embodiments, the phone 18 begins the sharing process on the occurrence of an event such as the user 10 entering the room containing the television 12 or on a request transmitted from the television 12 once the television 12 is turned on by the user 10.

In a fourth step 908, the phone 18 queries the coordinator status; more specifically, the phone 18 attempts to detect the presence of a coordinator, for example by detecting whether any of the devices described in the device control panel are currently connected and/or querying whether any devices are available as a coordinator.

If no coordinator is available, the phone 18 proceeds to a tenth step 920, where the phone 18 initiates a session with the phone 18 as a coordinator.

In the present example, the television 12 is available as a coordinator and therefore in a fifth step 910, the television 12 indicates that it is a coordinator.

In a sixth step 912, the phone 18 requests the sharing of data by the television 12.

If the sharing request is rejected, which occurs if the phone 18 does not have permission to receive information for the present context of the user 10 or if the phone 18 does not have permission to request data from the television 12, the phone 18 proceeds to the tenth step 920, where it initiates a session with the phone 18 as coordinator.

If the sharing request is accepted, in a seventh step 914, the television 12 shares data containing contextual information as has been described with reference to the third step 706 of FIG. 7.

Following the seventh step 914, in an eighth step 916, the television concludes the sharing session as has been described with reference to the sixth step 712 of FIG. 7.

In a ninth step 918, the television 12 ends the sharing process.

In the tenth step 920, the phone 18 initiates a session with the phone as coordinator.

The method as described with reference to FIG. 9 repeats as appropriate; for example following the transmission of data from the television 12 to the phone 18 and the ending of the sharing process by the television 12, the watch 16 can begin a data sharing process, query a coordinator status and receive an indicator that the phone 18 is available as a coordinator before requesting data from and receiving data from the phone 18. By transferring coordinator status between devices, data can be transmitted after the device that originally contained that data has ended the data sharing process. As an example, via the phone 18, the watch 16 can receive data on television viewing habits even after the television 12 has ended the data sharing process.

While the television 12 has been described as a coordinator (transmitting information) and the phone 18 has been described as receiving information, it will be appreciated that either device could be the coordinator or the receiver. More generally, any device may be a coordinator, a receiver, or both a coordinator and a receiver and the television 12 and the phone 18 are typically adapted to both send and receive data from each other. This enables devices to benefit from the sensors and capabilities of other devices. As an example, the watch 16 might comprise a heart rate monitor that can be used to determine the stress level of the user; the television 12 is unlikely to comprise such a heart rate sensor. By sharing contextual information with the watch 16, the television 12 is able to assess a user's stress level and customise program suggestions accordingly.

The sixth step 912 (requesting the sharing of data) typically requires the user 10 to indicate whether or not the sharing request should be accepted if the devices have not previously shared contextual information. This comprises the user setting up a device profile using the device control panel 400 the first time that a device is used to share information. Thereafter, requesting the sharing of data occurs solely between the devices (without further input from the user 10). Where sharing is requested for a context and/or a device that has not been set up appropriately, the user 10 typically receives a prompt (e.g. a notification on the television 12 that reads “enable sharing with MyPhone?”); the user is then able to accept or reject the sharing request. This choice can be used only in relation to that specific request or it can be recorded in the context control panel 300, the device control panel 400 and/or the database 500.

While FIG. 9 has been described with reference to sharing between two devices, a similar process may be performed between applications on the same device. The phone 18 comprises a plurality of applications between which contextual information can be shared. As an example, the phone 18 may contain a fitness application and a recipe application. Using the method described with reference to FIG. 8, the fitness application and the recipe application are able to share contextual information. The fitness application is adapted to measure the heart rate of the user 10; this heart rate is useable to determine that the user 10 is exercising and this is shared with the recipe application to suggest suitable post-exercise meals.

Where contextual information is shared, either between applications on the same device or between separate devices, battery and processing power may be saved by not reusing sensors. Where a first application on the phone 18 obtains location data from a second application on the phone 18, there is no need for the first application to interact with a GPS sensor of the phone 18, which would drain battery.

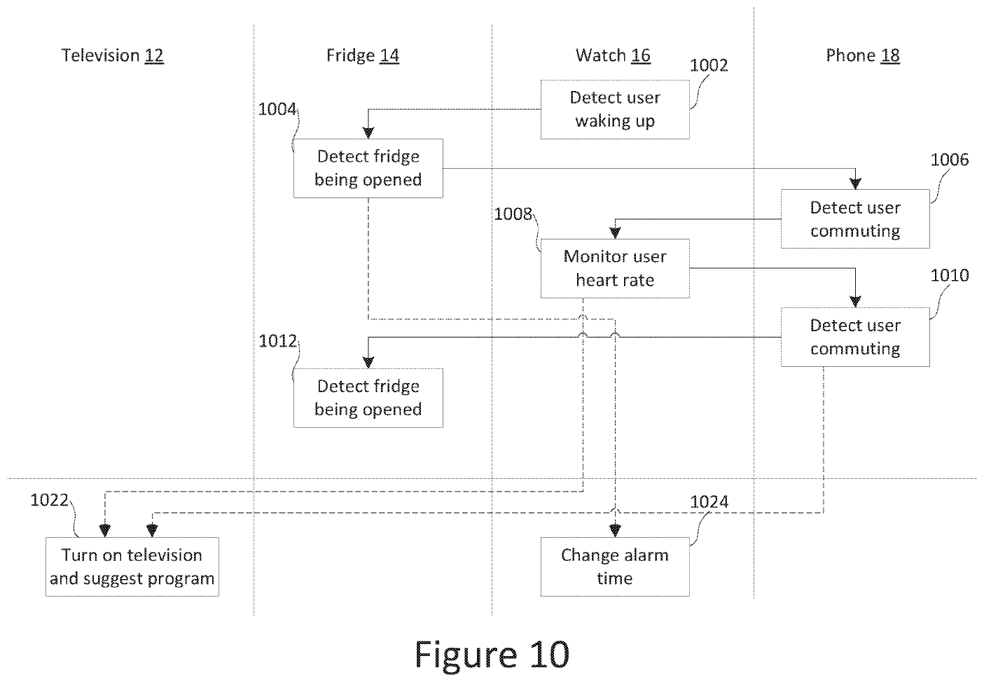

Referring to FIG. 10, there is shown an exemplary flowchart for how the devices 12, 14, 16, 18 detect information during a time period and use this to customise operations.

In a first step 1002, the user 10 waking up is detected by the watch 16. In a second step 1004, the user 10 opening the fridge is detected by the fridge 14. In a third step 1006, the user 10 commuting is detected by the phone 18. In a fourth step 1008, the heart rate of the user 10 is monitored by the watch 16. In a fifth step 1010, the user 10 commuting is detected by the phone 18. Finally, in sixth step 1012, the user opening the fridge is detected by the fridge 14.

These steps relate to a subset of actions taken by the user during a typical day—waking up, making breakfast, commuting to work, spending time at work, commuting home, and making dinner. The combination of devices 12, 14, 16, 18 are adapted to monitor the behaviour of the user 10 during this time and together are able to assess the user's situation and perform operations dependent on this situation.

Exemplary operations include turning on the television and suggesting a program 1022 when the user returns home. Presently, viewers of television turn on their televisions and select a program to watch based on their mood. Certain televisions are presently able to suggest programs; however, these programs are based on historic viewing habits only and do not take into account the context, e.g. the mood, of the viewer—and current televisions do not have a way to assess this context.

The present disclosure relates at least in part to a method of a device performing such an assessment. The television 12 shares data with the watch 16 and the phone 18 as described with reference to FIG. 8. Specifically, the television 12 receives heart rate information from the watch 16 and commute data from the phone 18. This information is usable to determine an estimated arrival time and a measure of stress. The television 12 is adapted to turn on based on the arrival time and to customise a program suggestion based on the arrival time and stress rate of the user 10—for example if the user 10 has had a stressful day, relaxing music and/or relaxing television programs are played and/or suggested.

In some embodiments, the watch 16 and/or the phone 18 analyses the raw data before transmission to the television. More generally, the device that processes the raw data typically depends on the capabilities of each device—devices with powerful processors may perform data analysis for information measured by other devices with less powerful processors. In this example, the heart rate data may be transmitted from the watch 16 to the phone 18, analysed by the phone 18 to obtain a measure of stress, and this measure of stress then transmitted from the phone 18 to the television 12. This separation of measurement and analysis is also useable to combine data before analysis (e.g. so that both the commute data and the heart rate data are considered in obtaining the measure of stress) and to protect data (e.g. to ensure that data is only stored/analysed on trusted devices).

In another example, the watch 16 is adapted to change an alarm time 1024 in dependence on the fridge being opened. The fridge 14 detects whether or not the fridge 14 is opened each morning—and if the user 10 does not open the fridge 14 in the morning, it might be inferred that the user has woken up too late to make breakfast and so an alarm might be set for an earlier time the next morning. In some embodiments, the fridge 14 shares data with the watch 16 and the phone 18 to obtain a historic baseline for the behaviour of the user 10. If it is detected, for example, that the user 10 has only recently begun checking emails in the morning and also that the fridge is not being used, the watch 16 and/or phone 18 might suggest setting an earlier alarm and indicate to the user 10 that this is to allow for time to check emails. Similarly, the phone 18 may share a holiday schedule with the fridge 14 so that for the duration of a holiday the fridge 14 does not associate not being opened with the user 10 getting up late.

Another exemplary use case is a smart speaker personalisation based on the events of the user's day and the user's current mood. This use case requires the watch 16, the phone 18, a smart door lock (not shown), smart lights (not shown), and smart speakers (not shown). This use case is described below:

The user 10:
wakes up at 06:00 on a Thursday morning;
goes to take a shower;
gets dressed and checks the phone 18 for any e-mails;
has a quick breakfast and leaves home by car;
gets to work around 08:30;
has their first meeting at 10:00 in the same building;
grabs lunch from the deli across the road around 12:30;
leaves the office around 15:30 for a 16:00 customer meeting;
gets stuck in traffic while driving on the motorway;
becomes stressed that they'll be running late for the meeting;
eventually get to the meeting 15 mins late, stressed;
attends the meeting until around 17:30;
leaves for home, right in the middle of rush hour and is stuck in traffic for 1.5 hours; and
eventually gets back home, tired and stressed around 20:00. Each of these actions is detected by the watch 16 and/or the phone 18.

When the user 10 gets home, data from the phone 18 is transmitted to the smart door lock, for example via a Bluetooth® network; the data comprises contextual information relating to the location of the user 10. Following receipt of this information, the smart door lock unlocks.

As the user 10 walks into their home, data from the watch 16 and phone 18 is transmitted to the lighting system and the smart speaker, for example via Wi-Fi. The data comprises contextual information that indicates the user 10 has had a stressful day; based on this contextual information the speaker begins to play calming music and the lighting system changes to an appropriate colour.

Further exemplary use cases include:

Smart speakers (playing music based on the context information transferred from the watch 16 and/or phone 18 without requiring any audio input from the user 10. As a result, when the user 10 enters their home, their speakers already know what music to play based on how their day has been).
Smart TV (understanding whether it is a group of users watching TV or just the user 10 which can then be used to tailor the recommendations).
Smart advertising: understanding the context of the user 10 to tailor advertising. For example, if the user 10 has recently finished exercising, fitness advertisements are shown, e.g. for supplements.
Gateless ticketing at train stations and on-board buses (trusted device interaction allows a passenger to automatically board and leave a train/bus without the need for physical barriers).
Smart recommendations (based on the context history being shared between devices, the correct content length can be predicted to then be used in recommendations. For example, if the user's commute length is predicted to be 20 minutes, a video recommendation app should not show any videos that are greater than 20 minutes).
Determining how people arrive at a concert: understanding how people arrived at a concert (e.g. walk, bus, car) could be used to determine how much traffic to expect at the end of the concert, where the most traffic build-up will be, and if any additional adverts can be placed along those journey points to upsell merchandise. The contextual information can be gathered on the user's smartphone and then shared when they check in to the concert arena.
Smart food delivery updates: if the user 10 is working late at the office and wants to place a food order to be delivered to his home shortly after he arrives, updated contextual information can be sent to the food delivery service's online portal such that when he is about 20 minutes away from home they send the delivery, and not before that. This live context update ensures that the user 10 will be at home when the food is delivered and that the food is warm on delivery, making for a pleasant eating experience.
Smart cabs: before getting in a cab, if the user's prior journey is known (especially the duration and type) by knowing their contextual history, this could be helpful to provide a personalized cab experience. For example, if the user 10 has had a long day in meetings and is quite stressed out, this information could be useful for the cab driver (or autonomous vehicle) to play calming music and avoid any unnecessary conversation with the customer. This would ensure that the user 10 has a pleasant and uneventful journey. Or the user 10 could be running late for a meeting in which case the cab driver might want to take the fastest route to the destination.
Smart appliances, such as kettles or toasters: if the location, situation, and mood of the user 10 is inferred, appliances can be set to prepare food that suits the circumstances of the user that will be ready at a set time, e.g. coffee that is ready when the user gets out of bed in the morning. A waking up time is inferred, e.g. from a sleep time and a daily schedule.

### Alternatives and Modifications

The contextual information can be shared either from device to device (e.g. client to client) or between devices via a server (e.g. client to server).

Typically, one or more of the computer devices on which the disclosed methods are implemented is arranged to share contextual information directly with another computer device that is arranged to carry out operations based on contextual information. In some embodiments, this sharing directly between devices depends on proximity, where for example sharing directly between devices may require a Bluetooth® connection and/or each device to be connected to the same local area network.

The sharing of contextual information directly between devices occurs as has been described in the detailed description as above. The sharing may depend on the contextual information that each device is permissioned to access, where the sharing device may request relevant permissions from the requesting device before sharing any contextual information.

The sharing of information solely between devices may be used in only certain circumstances, or only with certain types of contextual information. This may be used to ensure that sensitive contextual information is only ever accessed by selected computer devices.

Typically, information (e.g contextual information and/or sensor data) is also shared between devices via a server, e.g. a ‘cloud’ server that connects each device via the Internet. Typically, the server is not arranged to carry out operations based on the contextual information; instead the server is arranged only to store and communicate the contextual information. The server may also be arranged to infer the contextual information.

Typically, all contextual information is shared with the server—as has been described in the detailed description above—where devices accessing the information stored by the server are required to prevent relevant permissions to the server in order to access the stored contextual information.

Each device may be arranged to communicate with other devices where possible (e.g. where there is a shared local area network) and arranged to communicate with a server where direct communication is not possible (e.g. because another device is turned off).

Furthermore, devices may be arranged to share between devices via one or more intermediary devices; this enables the sharing of sensor data and contextual information where the two devices that are not in range of each other but are both in range of an intermediary device. In this situation, the sharing of contextual information and/or sensor data may depend only on the permissions of the two end devices (and the transmitted signals may be encrypted so that the intermediary device cannot access sensitive information), or it may also depend on the permissions of the intermediary device.

While the detailed description has referred to ambient devices being fixed in place and portable devices being movable, it will be understood that any of the described devices may be moveable or fixed in place. The method described is applicable to any combination of devices whether they are ambient, portable, fixed in place, or moveable.

While the detailed description has primarily described the sharing of data between the television 12 and the phone 18, it will be appreciated that any steps of the described method may be performed by any device.

In some embodiments, each device shares data with a “master” coordinator—and the master coordinator stores a database as has been described with reference to FIG. 5. This master coordinator is typically a computer device 2000 that has a large storage 2008 and/or a powerful CPU 2002. This enables data to be stored and analysed securely and quickly. In some embodiments, the master coordinator comprises a cloud server; this ensures that data is not lost if a device runs out of battery or breaks. The master coordinator may always be on and in a connected state (so that it is always available to share data). This enables devices with smaller storage to transmit data to the master coordinator at any time—and then delete the data.

In various embodiments, various communication protocols are used to share data, for example: Wi-Fi; Bluetooth®; cellular networks; radio frequency identification (RFID); LoRA; sound; Li-Fi; Near Field Communication (NFC); local area networks; wide area networks; and the internet.

Contextual information may also comprise:

| Context Type | Examples |
|---|---|
| Device Motion | Moving, Not Moving |
| Device Position | In Hand, In Pocket, In Bag, On Surface, |
| | On Arm, Against Ear |
| Device Orientation | Facing Down, Facing Up, Pointing Down, |
| | Pointing Up, Sideways |
| Device Type | Smartphone, Tablet, Smartwatch, Glasses, |
| | Shoes, Activity Tracker |
| Calendar information | Number of meetings, meeting types |
| | (personal/work-related), saved events. |

Contextual information may also be used for the operation of smart door locks (e.g. unlocking doors if a specific device is recognised).

Embodiments of the disclosure herein variously comprise any, some, or all of the following features, in any appropriate combination:
Sharing contextual information between devices.
Inferring contextual information from (raw) data.
Contextual information relates to a mood and/or stress level (or e.g. A mindset)—not something physical/past actions.
Contextual information is inferred from more than one piece of information.
Information is obtained by a number different of sensors and/or devices.
Information is obtained by a single device.
Contextual information is not directly related to the operation being performed (e.g. Music played depends on mood, not on past music played).
Contextual information is received from another (separate) device.
Contextual information is inferred from data that is not normally obtainable (e.g. A speaker cannot normally obtain heart rate data).
Contextual information is received indirectly (all devices update a contextual database that can thereafter be queried).
Sharing permission depends on the context of the user and/or the contextual information type.
Sharing between personal devices and ambient devices.
Personal devices have contextual information taken throughout a user's day and from a user's person that ambient devices do not normally have access to.
Contextual information and non-contextual information are both shared and combined to give a recommendation.
Learning from contextual information in order to make predictions in the future.
Sharing contextual information occurs based on an event occurring (e.g. User returning home).
The event is based upon contextual information.
The event is triggered by contextual information exceeding a threshold value (e.g. The user having a high stress level).
The event is triggered by the sharing device (not the receiving device).
Sharing occurs without input from the user.
Forming a context history based on contextual information.
Forming a baseline value.
Sharing contextual information when recent data varies substantially from the baseline value (e.g. Changing the status between discrete moods).
Personalizing the baseline value to relate to the user.
Having a coordinator and a receiver.
The coordinator being detected based upon already being connected.
A joining device joining as either a coordinator or a receiver depending on whether a coordinator is already present.
A coordinator handing coordinator status down to another device if/when it stops data sharing.

It will be understood that the present invention has been described above purely by way of example, and modifications of detail can be made within the scope of the invention.

Reference numerals appearing in the claims are by way of illustration only and shall have no limiting effect on the scope of the claims.

1. An apparatus for sharing data, the apparatus comprising:
a communication interface for receiving data signals; wherein the data signals include a plurality of different types of contextual information relating to a user, wherein each type of contextual information comprises information inferred from sensor data; and
a processor for performing an operation in dependence on the plurality of different types of contextual information.

2. An apparatus according to claim 1, wherein the processor is arranged to determine whether the apparatus has permission to share the contextual information.

3. An apparatus according to claim 2, wherein determining whether the apparatus has permission to share the contextual information comprises determining one or more of:
whether the device has permission to: access, receive, and/or infer, a type of contextual information; and
determining a permission from a database of permissions; and
evaluating one or more of: previous permissions granted by the user; a user input; the type of contextual information being requested; the location of the apparatus; the time; a network connection status of the apparatus; a certificate held by the apparatus; the type of the apparatus; and whether the apparatus is part of a list of approved apparatuses.

4.-6. (canceled)

7. An apparatus according to claim 1, wherein the processor is arranged to determine one or more of: the types of contextual information that can be shared; the timeframes during which contextual information can be shared; the distance at which a device is allowed to share contextual information; the group of devices with which sharing of contextual information is allowed; and activities during which sharing of contextual information is allowed.

8. An apparatus according to claim 1, wherein the processor is arranged to determine a receiving apparatus, the receiving apparatus being arranged to receive data, and/or wherein the processor is arranged to determine a coordinating apparatus, the coordinating apparatus being arranged to transmit data.

9. (canceled)

10. An apparatus according to claim 1, wherein the processor and/or the communication interface is arranged to:
querying a connection status relating to one or more further apparatuses transmitting the contextual information;
determine a time at which the or each further apparatus initiated a data sharing session;
designate an application that first initiated the data sharing session as the coordinating application; and
determine a/the receiving apparatus and/or a/the coordinating apparatus based on at least one of: a type of the apparatus; a type of contextual information being requested; a location of the apparatus; a time; a network connection status of the respective apparatuses; a certificate held by one of the apparatuses; and whether the or each apparatus is on a list of approved apparatuses.

11.-13. (canceled)

14. An apparatus according to claim 1, wherein the processor and/or the communication interface is arranged to determine the status of a further apparatus; and join the apparatus to a data sharing session as either a coordinating apparatus or a receiving apparatus in dependence on the status of the further apparatus.

15. (canceled)

16. An apparatus according to claim 1, wherein the processor and/or the communication interface is arranged to:
join the apparatus to a data sharing session; and/or
join the apparatus to a data sharing session as a receiving apparatus when a/the further apparatus is available to share data received data signals comprise; and/or
join the apparatus to a data sharing session as a coordinating apparatus when a/the further apparatus is not available to share data received data signals; and/or
determine the status of a further apparatus, wherein the processor and/or the communication interface is adapted to determine at least one of: the availability of the further apparatus to share data; the type of device on which the further apparatus is implemented; the communication capability of the further apparatus; and a permission related to the further apparatus; and/or
leave the data sharing session; and designate the further apparatus as a/the coordinating application in dependence on the status of the apparatus.

17.-18. (canceled)

19. The apparatus of claim 1, wherein the communication interface is arranged to receive data signals via an intermediary apparatus and/or to receive data signals via an intermediary device wherein the apparatus is not in range of a sharing apparatus.

20. The apparatus of claim 0:
wherein the data signals are encrypted so as not to be accessible from the intermediary apparatus; and/or
wherein the processor is arranged to determine a permission of the intermediary apparatus.

21. (canceled)

22. An apparatus according to claim 1 wherein the contextual information comprises one or more of:
information relating to an attribute of the user;
information relating to at least one of: a physical attribute of the user; and a psychological attribute of the user;
an attribute of the user relating to a recent time period;
an attribute of the user from a day before receipt, and/or an hour before receipt, and/or 30 minutes before receipt, and/or a minute before receipt; and
information inferred from one or more of: a plurality of data; a plurality of types of data; and a plurality of data from different sources.

23.-27. (canceled)

28. An apparatus according to claim 1, wherein the communication interface is adapted to obtain data from a database comprising contextual information.

29. An apparatus according to claim 1, wherein the processor and/or the communication interface is arranged to determine a permission relating to the apparatus wherein:
the permission indicates whether the apparatus is permitted to receive and/or access a type of contextual information and/or sensor data; and/or
the processor and/or the communication interface is arranged to determine a plurality of permissions, wherein each permission relates to a different type of contextual information and/or sensor data; and/or
the permission indicates whether the apparatus is permitted to infer a type of contextual information.

30.-32. (canceled)

33. An apparatus according to claim 1, wherein the processor and/or the communication interface is arranged:
forming a context history; and/or
forming a context history based on the contextual information in the received data signals; and/or
receive further data signals, the further data signals containing historic contextual information; and form a context history based on the historic contextual information;
and/or evaluate the received contextual information in the received data signals in dependence on a/the context history.

34.-35. (canceled)

36. An apparatus according to claim 1, wherein the processor is arranged determine a baseline value for a type of contextual information relating to the user; and determining a difference between a recent value for the type of contextual information and the baseline value; wherein performing an operation in dependence on the contextual information comprises performing an operation in dependence on the determined difference.

37. An apparatus according to claim 1, wherein the communication interface is adapted to receive data signals in dependence on one or more of:
the occurrence of an event;
a change in a value relating to contextual information being detected; a value relating to contextual information exceeding a threshold value; and a value relating to contextual information falling below a threshold value; and
the event occurrence of an event externally to the apparatus.

38.-40. (canceled)

41. An apparatus according to claim 1, the processor and/or the communication interface is arranged to:
receive non-contextual information;
determine a non-contextual operation in dependence on the non-contextual information; and
modify the non-contextual operation in dependence on the contextual information.

42.-47. (canceled)

48. A system for sharing data, the system comprising:
a first application; and
a second application;
wherein the first application comprises:
a communication interface for receiving data signals from the second application, wherein the data signals contain a plurality of different types of contextual information relating to a user, wherein each type of contextual information comprises information inferred from sensor data; and
a processor for performing an operation in dependence on the plurality of different types of contextual information; and
wherein the second application comprises:
a communication interface for transmitting data signals to the first application.

49.-51. (canceled)

52. A method of sharing data, the method comprising:
receiving data signals, wherein the data signals contain a plurality of different types of contextual information relating to a user wherein each type of contextual information comprises information inferred from sensor data; and
performing an operation in dependence on the plurality of different types of contextual information.

53.-64. (canceled)
